(19) United States
(12) Patent Application Publication
(10) Pub. No.: US 2025/0255419 A1
(43) Pub. Date: Aug. 14, 2025

(54) DETERMINING MOVEMENT OF AN INDIVIDUAL ON A MATTRESS FROM HUMIDITY AND OTHER MEASUREMENTS

(71) Applicant: Purple Innovation, LLC, Lehi, UT (US)

(72) Inventors: Thomas Andrew Bennett, Draper, UT (US); Wade Henderson, Orlando, FL (US); Alex DuBois, Green Cove Springs, FL (US)

(21) Appl. No.: 18/441,516

(22) Filed: Feb. 14, 2024

(51) Int. Cl. A47C 31/12; A47C 21/04; A61B 5/00

(52) U.S. Cl. CPC A47C 31/123; A47C 21/044; A61B 5/4815; A61B 2562/029

(57) ABSTRACT

An apparatus that monitors activity during sleep employs one or more humidity sensors. The apparatus may include a sleeping surface, at least one humidity sensor in association with the sleeping surface, and at least one processor in communication with the at least one humidity sensor. The apparatus may monitor a humidity at the sleeping surface over a duration of time to determine one or more of a presence of an individual on the sleeping surface, a position of the individual on the sleeping surface, and/or movement by the individual on the sleeping surface, as well as a type of movement by the individual (e.g., a normal change in position, movement indicative of restless sleep, etc.). The apparatus may control adjustment of one or more characteristics of a cushion with which the sleeping surface is associated based on a type of movement detected and determined by the apparatus. Methods for monitoring activity during sleep and, optionally, adjusting the sleeping surface are also disclosed.

### TECHNICAL FIELD

This disclosure relates generally to apparatuses and methods for monitoring activity during sleep and to sleeping systems. More specifically, this disclosure relates to apparatuses that employ humidity sensors to detect an individual's location on a mattress and/or movement of an individual on the mattress, as well as to methods for using humidity measurements to detect an individual's location and/or movement by the individual and sleeping systems in which such information may be used to change a characteristic of a mattress.

### RELATED ART

A common issue while sleeping is the sleep environment and an individual, or a sleeper, being too hot or too cold as they sleep. Generally, a viable solution for when a sleeper is too cold is to add more blankets or layers to the sleep environment. When the sleep environment gets too hot, humidity is generally trapped between the sleeper and the mattress or bed they are sleeping on. However, there are generally not viable solutions for when a sleeper is too hot. While removing layers from the sleep environment may alleviate some of the built-up heat or humidity, simply removing layers does not proportionately remove heat or humidity from the sleep environment, leaving hot sleepers hot.

### SUMMARY

Disclosed are devices, methods, and systems for monitoring activity as an individual rests on a mattress (e.g., while reclining, lying down, during sleep, etc.).

In one aspect, an apparatus that can detect a location and/or movement of an individual on a mattress (e.g., while reclining on the mattress, while lying on the mattress, during sleeping, etc.) may include a sleeping surface, at least one humidity sensor in association with the sleeping surface, and at least one processor in communication with the at least one humidity sensor. The apparatus may comprise a cushion. The cushion may comprise a cushion of the mattress, a cushion of a mattress topper, or the like. Alternatively, the apparatus may comprise a cover for the mattress (e.g., a mattress pad, etc.). The at least one processor may be programmed to monitor a humidity at one or more locations on the sleeping surface over a period of time and determine a location of an individual on the sleeping surface or movement by the individual on the sleeping surface based on one or more humidity measurements at one or more corresponding locations on the sleeping surface and/or a detected change in humidity at one or more locations on the sleeping surface. Optionally, the at least one processor may adjust one or more functions of the cushion based on one or more humidity measurements and/or upon detecting a change in the humidity at one or more locations on the sleeping surface.

A method of this disclosure may include detecting a location of an individual on the sleeping surface and/or detecting movement by the individual on the sleeping surface (e.g., as the individual reclines on the sleeping surface, as the individual lies on the sleeping surface, during sleeping, etc.). The method may be conducted with an apparatus having a sleeping surface, at least one humidity sensor in association with the sleeping surface, and at least one processor in communication with the at least one humidity sensor. The method may also include capturing a plurality of humidity measurements over a period of time and adjusting one or more characteristics of at least a portion of the cushion based on the plurality of humidity measurements. The humidity measurements may be obtained at one location on the sleeping surface or at a plurality of different locations on the sleeping surface. The humidity measurements may be used to determine the location of the individual on the sleeping surface. Changes in the humidity measurements at one or more locations on the sleeping surface over time may be used to track movement of the individual over time.

A sleeping system may include an apparatus (e.g., a cushion, a mattress cover, etc.) with a sleeping surface and at least one humidity sensor associated with the sleeping surface. In some embodiments, the apparatus may include a plurality of humidity sensors at different locations or regions of the sleeping surface (e.g., a head region, one or more intermediate regions (e.g., a torso region, a leg region, etc.), a foot region, etc., of the sleeping surface. The sleeping system may also include at least one processor in communication with the at least one humidity sensor, where the at least one processor may receive and process humidity measurements obtained with the at least one humidity sensor. The at least one processor may process the humidity measurements to determine a location of an individual on the sleeping surface. By processing humidity measurements obtained over time, the at least one processor may detect movement of the individual on the sleeping surface.

The sleeping system may further include at least one adjustment mechanism that functions under control of the at least one processor. The adjustment mechanism may adjust one or more characteristics of a mattress or the sleep environment based on the detected location and/or movement of an individual on the sleeping surface. Examples of adjustment mechanisms include apparatuses that adjust an orientation of the sleeping surface (e.g., an adjustable platform for the mattress, air bladders within the mattress, etc.), a firmness of one or more locations of the mattress (e.g., air bladders within the mattress, etc.), movement of the mattress (e.g., internally within the mattress, by way of a platform for the mattress, etc.), ventilation of the mattress or a portion thereof, a temperature of the mattress or a portion thereof, or the like.

Other aspects of the disclosed subject matter, as well as features and advantages of various aspects of the disclosed subject matter, should be apparent to those of ordinary skill in the art through consideration of the ensuing description, the accompanying drawings, and the appended claims.

### BRIEF DESCRIPTION OF THE DRAWINGS

In the drawings:

FIG. 1A schematically illustrates a top view of a sleeping surface and FIG. 1B schematically illustrates a top view of a sleeping system;

FIGS. 1C and 1D schematically illustrates a tip view of another embodiment of a sleeping surface and a top view of another embodiment of a sleeping system, respectively;

FIG. 2 schematically illustrates a side view of a smart mattress, which may include the sleeping surface of FIG. 1A;

FIGS. 3-4 are flowcharts of example methods for monitoring or detecting an individual's location and/or activity (e.g., movement) on a mattress; and

FIGS. 5A-5B graphically illustrate a detection of an individual's location and/or activity (e.g., movement) on a mattress.

### DETAILED DESCRIPTION

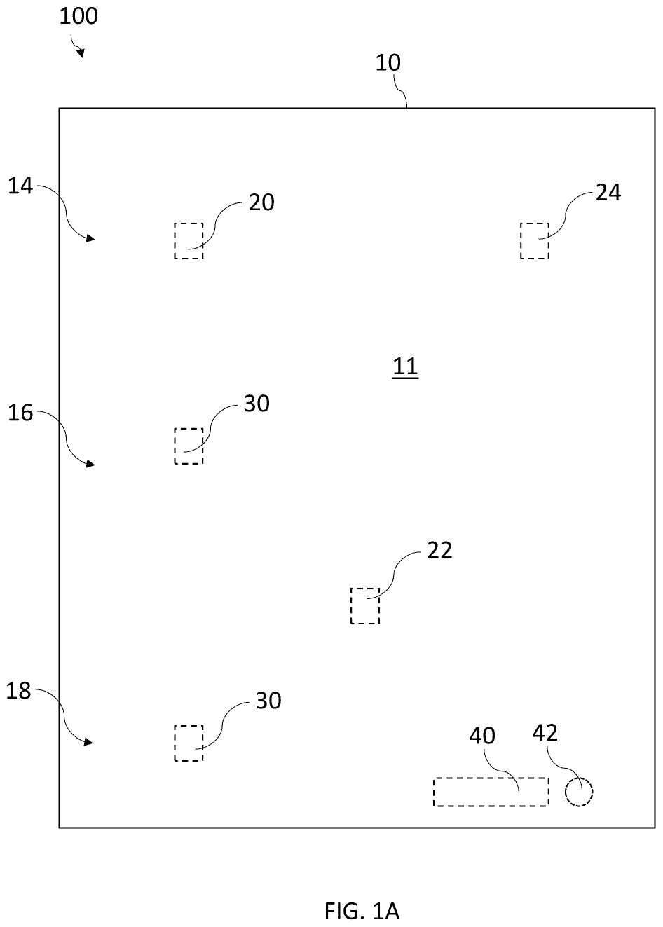

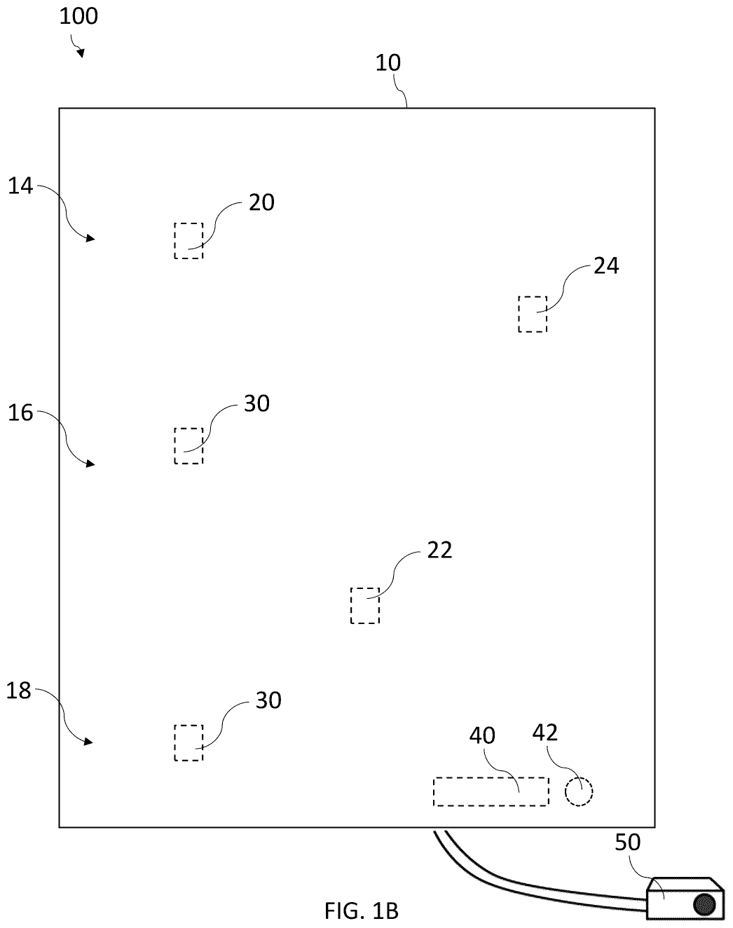

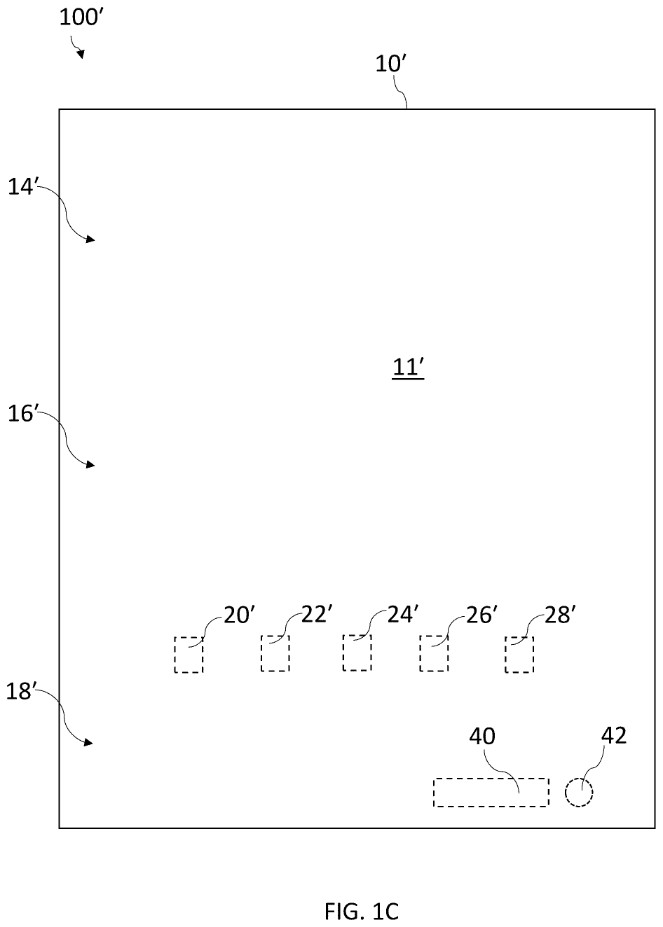

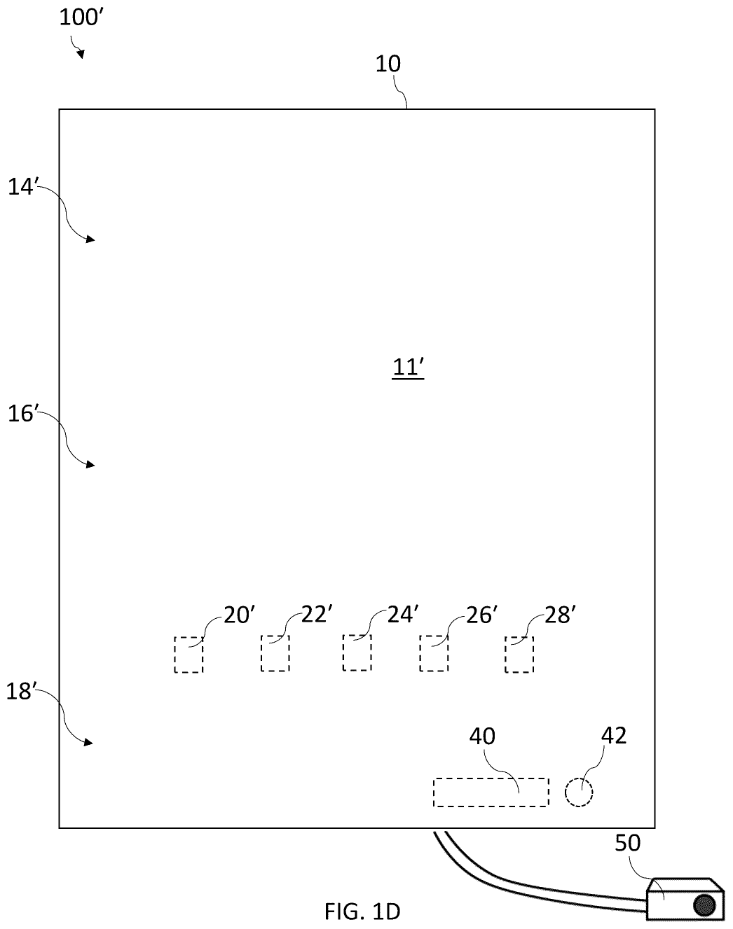

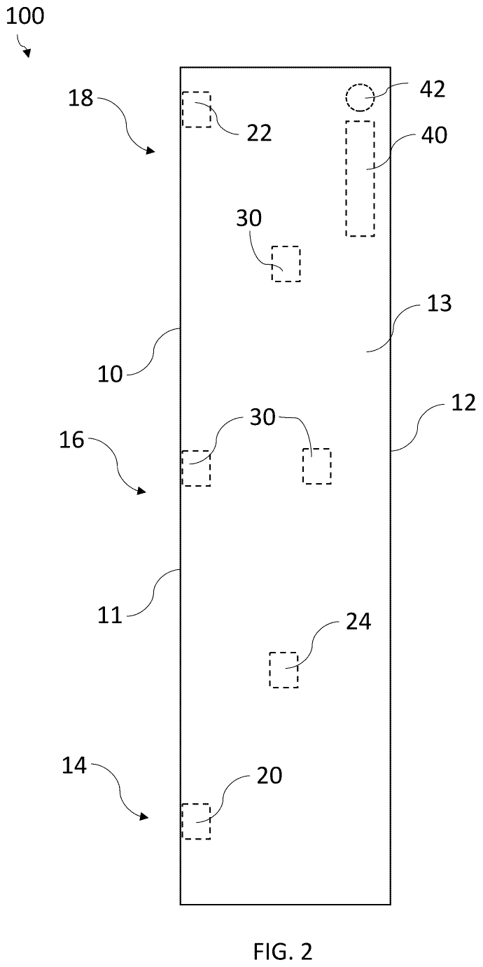

Referring to FIGS. 1A, 1B, and 2, an apparatus 100 includes a sleeping surface 11 and a base surface 12 opposite from the sleeping surface 11. In some embodiments, the apparatus 100 may comprise a cushion 10, such as a mattress, a mattress topper, or the like. Such an apparatus may include a cushion core 13 between the sleeping surface 11 and the base surface 12. In other embodiments, the apparatus 100 may comprise a device to be placed on a mattress or a mattress pad (e.g., a mattress pad, a mattress cover, etc.). A sleeping environment (not illustrated) may be formed by or defined between the sleeping surface 11 and any covers (e.g., blankets, sheets, etc.) over the sleeping surface 11. The sleeping environment may be where a user rests or sleeps or when reclining or lying on the sleeping surface 11. The sleeping environment may be monitored by one or more sensors 20, 22, 24, which may comprise humidity sensors, or other sensors 30 of other types carried by the cushion 100 in association with the sleeping surface 11 (see FIG. 2).

The sleeping surface 11 of the apparatus 100 may have a head region 14, which may also be referred to as a “first region,” at least one intermediate region 16, which may be referred to as a “second region,” and a foot region 18, which may be referred to as a “third region.” In some embodiments, the apparatus 100 is connectable to or associated with an adjustment mechanism 50. As illustrated in FIG. 1B, the adjustment mechanism 50 may include a ventilation system (e.g., a pump (e.g., a fan or vacuum) and associated wiring and conduits to connect the pump to a mattress or mattress topper (e.g., the cushion 10, etc.) or to a portion of the mattress or mattress topper. As explained more below, the adjustment mechanism 50 may alter or adjust one or more characteristics of a mattress or mattress topper (e.g., the cushion 10, etc.) with which the apparatus 100 is associated and/or one or more characteristics of the sleeping surface 11.

In embodiments where the apparatus 100 comprises a mattress or a mattress topper, the cushion 10 and/or the cushion core 13 may be at least partially constructed or formed from an elastomeric gel material. Examples of elastomeric gel materials that may be used to form the cushion 10 and/or the cushion core 13 are described by U.S. Pat. No. 5,994,450, titled “GELATINOUS ELASTOMER AND METHODS OF MAKING AND USING THE SAME AND ARTICLES MADE THEREFROM,” issued on Nov. 30, 1999; U.S. Pat. No. 6,413,458, titled “PROCESS FOR FORMING GELATINOUS ELASTOMER MATERIALS,” issued on Jul. 2, 2002; U.S. Pat. No. 6,797,765, titled “GELATINOUS ELASTOMER,” issued on Sep. 28, 2004; and U.S. Pat. No. 7,964,664, titled “GEL WITH WIDE DISTRIBUTION OF MW IN MID-BLOCK,” issued Jun. 21, 2011. The entire contents of each of the foregoing are herein incorporated by reference.

In some embodiments, the cushion core 13 includes one or more layers, such as those illustrated and described in U.S. Pat. No. 11,317,733, titled “MATTRESSES INCLUDING AN ELASTOMERIC CUSHIONING ELEMENT AND A POCKETED COIL LAYER AND RELATED METHODS,” issued on May 3, 2022 and U.S. Pat. No. 11,213,139, titled “COMPOSITE MATTRESSES WITH AIR CHAMBERS,” issued on Jan. 4, 2022. Examples of a smart sleeping system or smart cushion are described and illustrated in U.S. patent application Ser. No. 17/978,119, titled “SLEEP SEQUENCE INITIATION FOR A SMART BED,” filed on Oct. 31, 2022, and U.S. patent application Ser. No. 17/978,141, titled “SYNCHRONIZATION OF PRESSURIZATION WITH ARRANGEMENT OF ADJUSTABLE PLATFORM,” filed on Oct. 31, 2022. The entire contents of each of the foregoing are herein incorporated by reference.

The elastomeric materials (e.g., elastomeric gel materials, etc.) of the cushion 10 and/or the cushion core 13 may define a plurality of interconnected walls that form or define a plurality of hollow columns with voids. The plurality of hollow columns may allow for air to pass through the cushion 10, such as from the base surface 12, through the cushion core 13, and to the sleeping surface 11. Examples of cushions 10 and/or cushion cores 13 with interconnected walls that define hollow columns are described by U.S. Pat. No. 6,026,527, titled “GELATINOUS CUSHIONS WITH BUCKLING COLUMNS,” issued on Feb. 22, 2000; U.S. Pat. No. 7,060,213, titled “CUSHIONING DEVICES, GELATINOUS ELASTOMER MATERIALS, AND DEVICES MADE THEREFROM,” issued on Jun. 13, 2006; U.S. Pat. No. 7,076,822, titled “STACKED CUSHIONS,” issued on Jul. 18, 2006; U.S. Pat. No. 8,919,750, titled “CUSHIONING ELEMENTS COMPRISING BUCKLING WALLS AND METHODS OF FORMING SUCH CUSHIONING ELEMENTS,” issued on Dec. 30, 2014; and U.S. Pat. No. 11,793,322, titled “MATTRESSES AND MATTRESS TOPPERS INCLUDING KNITTED FABRIC, AND RELATED METHODS,” issued on Oct. 24, 2023. The entire contents of each of the foregoing are herein incorporated by reference.

Disposed throughout the cushion 10, if any, throughout the cushion core 13, if any, at the sleeping surface 11, and/or otherwise in association with the sleeping surface 11 (e.g., just above the sleeping surface 11 (e.g., between the cushion 10 and a cover therefor, within a cover for the cushion 10, etc.), on the sleeping surface 11 (e.g., on substrates placed on the sleeping surface 11, etc.), just beneath the sleeping surface 11 (e.g., in voids of the cushion 10, etc.), in communication with the sleeping surface 11 (e.g., in voids of the cushion 10, etc.) may be one or more sensors 20, 22, 24, 30. The one or more sensors 20, 22, 24, 30 may include, for example, a first humidity sensor 20, a second humidity sensor 22, a third humidity sensor 24, and any of a variety of different types of sensors 30 (e.g., temperature sensors, pressure sensors, optical sensors, etc.). The one or more sensors 20, 22, 24, 30 may be positioned about the sleeping surface 11 at locations that correspond to the various regions (e.g., the head region 14, intermediate region 16, foot region 18, etc.) of the sleeping surface 11.

For example, the first humidity sensor 20 may be positioned in the head region 14 of the sleeping surface 11 and may monitor a humidity about the head region 14. In some embodiments, the head region 14 corresponds to the head region of a mattress or mattress topper (e.g., the cushion 10, etc.) or where an individual's head, neck, and shoulders are typically positioned when the individual lies on a mattress. In some embodiments, the first humidity sensor 20 and the third humidity sensor 24 may be positioned within or about the head region 14.

The second humidity sensor 22 may be positioned in the third foot region 18 of the sleeping surface 11 and may monitor a humidity about the foot region 18. The foot region 18 may correspond to a foot region of a mattress or mattress topper (e.g., the cushion 10, etc.), where the legs and feet of the individual are typically positioned when the individual lies on a mattress.

In some embodiments, the third humidity sensor 24 may be positioned between the head region 14 and the intermediate region 16 (i.e., near a transition between the head region 14 and intermediate region 16) and may monitor a humidity between the head region 14 and the intermediate region 16.

In some embodiments, another sensor 30, such as a pressure or temperature sensor, may be positioned in the intermediate region 16. The sensor 30 may monitor a temperature or a pressure about the intermediate region 16, which may correspond to the intermediate region of a mattress, where the individual will typically rest their the torso while lying on the sleeping surface of a mattress.

FIG. 1C shows an embodiment of a cushion 10′ that may be configured similarly to or the same as the embodiment of cushion 10 shown in and described with reference to FIGS. 1A, 1B, and 2. The cushion 10′ may comprise a mattress with a sleeping surface 11′. The sleeping surface 11′ may include a head region 14′, at least one intermediate region 16′, and a foot region 18′. Sensors 20′, 22′, 24′, 26′, and 28′ may be provided at or adjacent to the sleeping surface 11′. For example, the sensors 20′, 22′, 24′, 26′, and 28′ may be positioned on the sleeping surface 11′ (e.g., on a strip laid across the sleeping surface 11′, etc.). As another example, the sensors 20′, 22′, 24′, 26′, and 28′ may be positioned just above the sleeping surface 11′ (e.g., within a cover for the cushion 10′, between the cushion 10′ a cover for the cushion 10′, etc.). As yet another example, the sensors 20′, 22′, 24′, 26′, and 28′ may be positioned beneath the sleeping surface 11′ (e.g., within voids of the cushion 10′, etc.). As illustrated, the sensors 20′, 22′, 24′, 26′, and 28′ may be substantially aligned or aligned across at least a portion of the width of the sleeping surface 11′, for example, along a portion of the sleeping surface 11′ where an individual's legs and/or feet (e.g., thighs, knees, calves, ankles, feet, etc.) will rest as the individual sleeps. Thus, the sensors 20′, 22′, 24′, 26′, and 28′ may be positioned in an intermediate region 16′, between an intermediate region 16′ and the foot region 18′, or in the foot region 18′ of the sleeping surface 10′. FIG. 1D depicts an embodiment of a sleeping system that includes the embodiment of cushion 10′ shown in FIG. 1C, which additionally includes an adjustment mechanism 50, such as that described in reference to FIG. 1B.

Referring to FIG. 2, the sensors 20, 22, 24, 30 (or the sensors 20′, 22′, 24′, 26′, and 28′ of the embodiment of cushion 10′ shown in FIGS. 1C and 1D) may be positioned at any of a variety of depths relative to the sleeping surface 11. For example, the first humidity sensor 20 may be positioned at, adjacent to, or just under the sleeping surface 11 in approximately the head region 14. The second humidity sensor 22 may be positioned at, adjacent to, or just under the sleeping surface 11 in approximately the foot region 18. In some embodiments, another sensor 30 (e.g., a temperature sensor, a pressure sensor, etc.) may be positioned beneath the sleeping surface 11 (e.g., within the cushion core 13, etc.).

Also associated with the apparatus 100 (e.g., with a cushion 10 of the apparatus 100, etc.) may be a processor 40 and a transceiver 42 (which may, optionally, include both a transmitter and a receiver). In some embodiments, the processor 40 and the transceiver 42 are carried by the apparatus 100, though the processor 40 and the transceiver 42 may be remote from the apparatus 100. The processor 40 may be in communication (e.g., electrical) with the one or more sensors 20, 22, 24, 30. The processor 40 may receive measurements taken by the one or more sensors 20, 22, 24, 30 and may be programmed to monitor and analyze the received measurements over time. For example, the processor 40 may receive, monitor, and analyze the received measurements for a period of time, such as 5 minutes, 30 minutes, 1 hour, 2 hours, 8 hours, 24 hours, multiple days, or more. The transceiver 42 may facilitate the transmission of data obtained with the one or more sensors 20, 22, 24, 30 to the processor 40.

Based on the received and analyzed data, the processor 40 may determine a humidity. The humidity determination may be correlated to or associated with a presence of an individual on the sleeping surface, a location of the individual on the sleeping surface 11, and/or movement of the individual on the sleeping surface 11. The presence (or absence) of an individual on the sleeping surface 11 may be determined by detecting an above-ambient humidity with at least one sensor 20, 22, 24. The location of the individual on the sleeping surface 11 may be determined by comparing a measured humidity at one or more sensors 20, 22, 24 with an ambient humidity and with historical (immediately prior, recent, past, etc.) humidity measurements as the individual lies on the sleeping surface 11. Movement of the individual may be determined by detecting changes in humidity at one or more sensors 20, 22, 24 while the individual lies on the sleeping surface 11 (e.g., a number of quick changes in humidity; a lesser number of more gradual changes in humidity, no change in humidity, etc.) over time. The extent of the change may be significant enough to represent a change in distance of an individual's body from a sensor 20, 22, 24. Without limitation, a change in the humidity detected by a sensor 20, 22, 24 of at least 2%, at least 3%, at least 5%, or at least 10% may represent movement of the individual relative to the sensor 20, 22, 24.

The processor 40 may determine a presence of an individual on the sleeping surface 11 by nature of the processor 40 receiving data indicative of above-ambient humidity from one or more of the sensors 20, 22, 24. The processor 40 may also determine a location of the individual on the sleeping surface 11. For example, increased humidity measurements obtained by the sensor 20 at the head region 14 and the sensor 22 at the intermediate region 16, but not by the sensor 24 at the foot region 18 may correspond to an individual occupying the head region 14 and intermediate region 16, but not the foot region 18. This may be indicative of a smaller sized individual (e.g., a child, etc.), an individual sitting up in bed, or the sleeping position of the individual (e.g., curled up, etc.). In some embodiments, the presence of a user of the cushion 10 and/or the sleeping surface 11 may be associated with a sharp increase or spike in humidity measured (e.g., from an ambient humidity (e.g, about 45% relative humidity, etc.) to an above-ambient humidity (e.g., about 50% or more humidity).

A number of quick, significant changes in humidity (i.e., humidity spikes) over a short period of time (e.g., less than a second, a second or two, a few seconds, a minute, a few minutes, etc.) may be associated with restless sleeping (e.g., tossing and turning movements, restless legs, spasms, etc.) during sleep. Specifically, the number of changes in humidity may occur because humidity gets trapped over the sleeping surface 11 by an individual's body and quick, repetitive changes in the individual's sleeping position repeatedly changes humidity over the sleeping surface 11. Thus, when the user repeatedly changes position over a short period of time (e.g., while tossing and turning, during restless leg movement, during spasms, etc.), the amount of humidity detected by the one or more sensors 20, 22, 24 quickly changes (e.g., the trapped humidity is suddenly released, the humidity level quickly rises, etc.). For example, quick, repetitive changes in the individual's sleeping position may release humidity from the sleeping surface 11 by moving any bed linens, blankets, etc. (i.e., covers) from a portion of the sleeping surface 11 or by forcing the humidity from beneath any covers over the individual. As another example, quick, repetitive changes in the individual's sleeping position may generate humidity, increasing humidity over the sleeping surface 11. Repeated, quick changes in humidity (e.g., the sudden release of humidity, the sudden increase in humidity, etc.) that represent restless sleep may occur over a short period of time, such as within a few minutes (e.g., 5 minutes, 10 minutes, etc.). For example, the relative humidity at time 1 may be measured at 60% and the humidity at time 2 may be measured at 55%, where time 2 is within a few minutes of time 1, representing restless sleep by the individual.

Slower, more gradual, and/or less frequent changes in humidity over time may be associated with a more gradual change in sleeping position (e.g., movement between a side-sleeping position and a back-sleeping position, movement of an individual's arms or legs, etc.). The more gradual change in sleeping position is a different type of movement than restless sleeping, which causes fewer changes in humidity over a given period of time and/or smaller changes in humidity. That is, the trapped humidity may be released gradually over an extended period of time (e.g., 20 minutes, 30 minutes, 45 minutes, 60 minutes, 90 minutes, etc.) when an individual changes sleeping position gradually or occasionally, as compared with when the individual sleeps restlessly.

By receiving and processing humidity data from one or more of the sensors 20, 22, 24, the processor 40 may monitor and/or detect an individual's location(s), movement(s) and/or activity during sleep, as well as determine the type of movements or activity (e.g., restless sleep, a change in position, etc.).

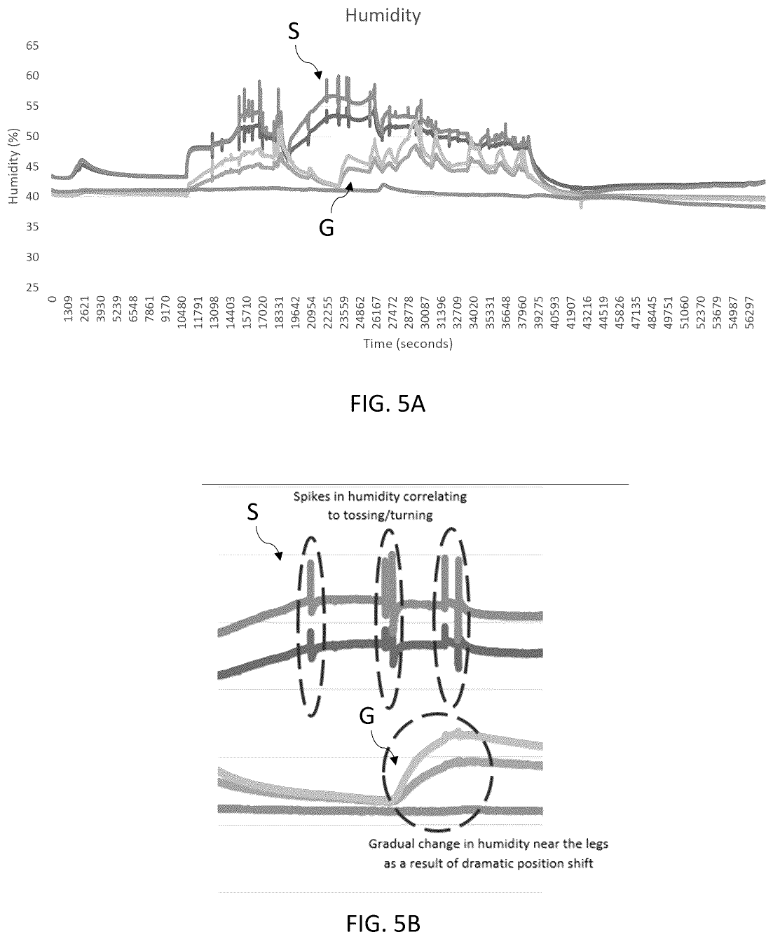

Referring to FIGS. 5A and 5B, illustrated are graphs showing example humidity measurements taken by one or more humidity sensors over time. The humidity measurements were obtained as individuals slept on a mattress having the configuration of the embodiment of cushion 10′ shown in FIG. 1C. FIG. 5A illustrates a plurality of humidity measurements taken over a period of about 15 hours and FIG. 5B illustrates a close-up of some of the humidity measurements from FIG. 5A. As clearly seen, a spike S in humidity is usually correlated to a sharp drop or sharp increase (e.g., a change of about 5% to about 15%) in humidity over a short period of time, such as within less than half an hour (30 minutes). The spikes S generally correspond to the types of movement that occur during restless sleeping (e.g., tossing and turning, etc.). In contrast, a gradual change G in humidity is usually correlated with slight changes in humidity over a longer period of time, such as within about one hour or more. The gradual changes G generally correspond to typical movements that occur during restful sleep (e.g., less frequent and/or more gradual changes in position, etc.).

The processor 40 and/or the transceiver 42 may be in communication (e.g., wired communication, wireless communication, etc.) with an adjustment mechanism 50, such that the processor 40 and/or the transceiver 42 can cause the adjustment mechanism 50 to adjust a characteristic of a mattress or mattress topper (e.g., the cushion 10, etc.) of which the apparatus 100 is a part or with which the apparatus 100 is associated based on the received data and processed humidity measurements. For example, when the processor 40 determines a spike in humidity has occurred at or near the sleeping surface 11, and the processor 40 has associated the spike in humidity with tossing and turning movements, the processor 40 may cause the adjustment mechanism 50 to adjust a flow of air to at least a portion of the sleeping surface 11. Additionally and/or alternatively, the processor 40 may cause the adjustment mechanism 50 to adjust a temperature of the sleeping surface 11, a contour of the sleeping surface 11, a firmness of the sleeping surface 11, etc., The transceiver 42 may also facilitate the transfer of commands to the adjustment mechanism 50 from the processor 40 (e.g., to adjust one or more characteristics of the mattress or mattress topper, etc.).

The one or more characteristics of the mattress or mattress topper that are adjusted may be intended to reduce a type of movement that has been detected by the processor 40. For example, the adjustment mechanism 50 may ventilate the sleeping surface 11, which can allow trapped humidity over the sleeping surface 11 to be released, thereby providing a more neutral sleeping environment for the user. “Neutral” may refer to a desired temperature and/or humidity for the sleeping environment and may be specific to the user. In other examples, regions of the sleeping surface 11 or the associated mattress or mattress topper can be selectively adjusted. For example, an orientation and/or contour of one or more of the head region 14, the intermediate region 16, and the foot region 18 may be adjusted by the adjustment mechanism 50.

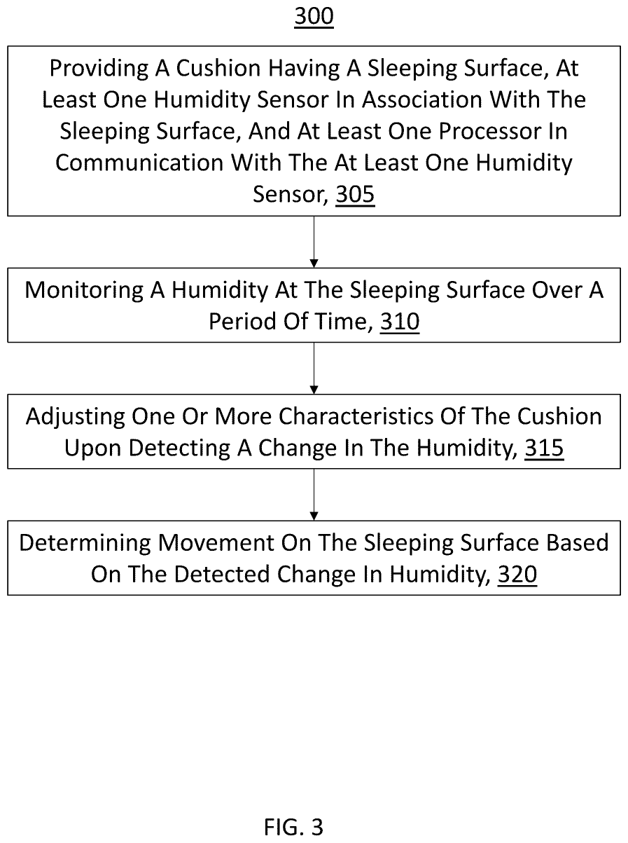

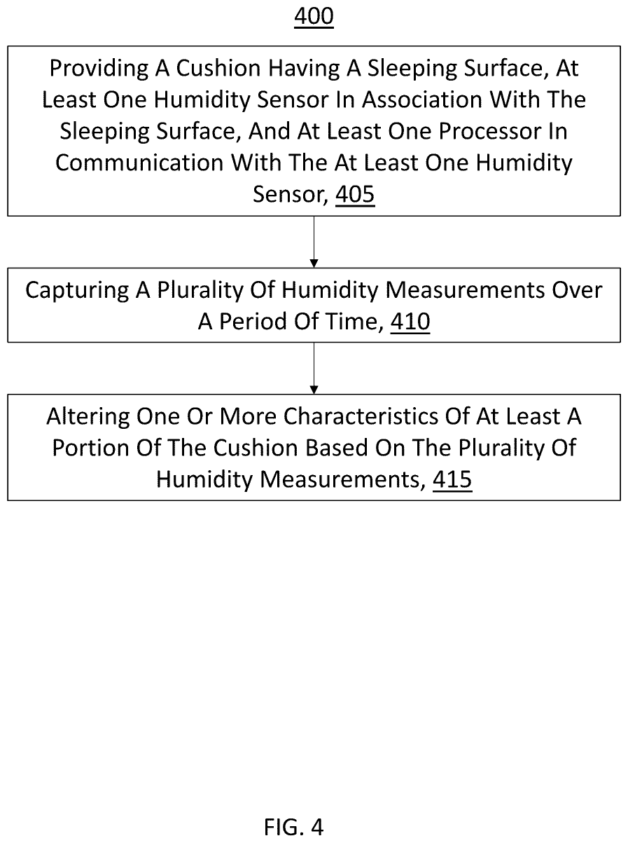

FIGS. 3 and 4 are flowcharts of example methods for monitoring or detecting activity (e.g., movement, etc.) of an individual on a mattress. In some embodiments, a method 300 may include providing an apparatus having a sleeping surface, at least one sensor that detects humidity in association with the sleeping surface, and at least one processor in connection with the at least one sensor, at 305. The apparatus may include or be associated with a mattress or a mattress topper (e.g., the cushion 10 of FIGS. 1A to 2, etc.). The at least one sensor may include a plurality of sensors distributed across the sleeping surface (e.g., the sleeping surface 11 of FIGS. 1A to 2, etc.) (e.g., about, in communication with, and/or beneath the sleeping surface in one or more regions of the sleeping surface; see FIG. 2).

The method 300 may also include monitoring a humidity at one or more locations of the sleeping surface over a period of time, at 310. For example, the at least one sensor may collect a plurality of humidity measurements while a user is resting or sleeping on the cushion (e.g., overnight, etc.). In some embodiments, the at least one sensor includes a plurality of sensors carried by the apparatus at various locations relative to the sleeping surface, each sensor collecting a plurality of humidity measurements. Monitoring a humidity at the sleeping surface may include monitoring a humidity at various regions of the sleeping surface. For example, the humidity at a first region may be monitored, where the first region may correspond to a position of an individual's head, neck, and shoulder. Additionally, the humidity at a second region may be monitored, where the second region corresponds to a position of the individual's torso, and/or the humidity at a third region may be monitored, where the third region corresponds to a position of the individual's legs and feet.

The method 300 may further include adjusting one or more characteristics of a mattress or mattress topper upon detecting a change in humidity, at 315. The processor may receive and analyze the humidity measurements obtained by the at least one sensor (or the plurality of sensors) and make a humidity determination based on the received and analyzed humidity data (e.g., a spike in humidity). That is, the processor may detect a change in humidity. The method 300 may include determining a location of an individual on the sleeping surface and/or detecting movement of the individual on the sleeping surface based on the detected change(s) in humidity. For example, when the detected change in humidity is a spike (e.g., a sharp drop and/or increase in humidity, etc.), the processor may determine that a user is tossing and turning upon the sleeping surface of the cushion and may adjust one or more characteristics of the mattress or mattress topper (e.g., ventilation, temperature, orientation and/or contour, firmness, etc.) to counteract or reduce such movement.

In some embodiments, a method 400 includes providing a cushion having a sleeping surface, at least one humidity sensor in association with the sleeping surface, and at least one processor in connection with the at least one humidity sensor, at 405. As before, the cushion may be the cushion 10 of FIGS. 1A to 2. The at least one humidity sensor may include a plurality of humidity sensors distributed throughout the cushion (e.g., about, in communication with, and/or and beneath the sleeping surface 11 in a head region 14, intermediate region 16, and/or foot region 18 of the cushion 10, see FIG. 2).

The method 400 may also include capturing a plurality of humidity measurements over a period of time, at 410. Also as before, the plurality of humidity measurements may be captured at a plurality of positions about the cushion 10 and/or the sleeping surface 11. The method 400 may further include adjusting one or more characteristics of at least a portion of the cushion 11 based on the plurality of humidity measurements, at 415.

For example, based on the plurality of humidity measurements, the processor may determine that a spike in humidity has occurred and correlated that spike in humidity with a user tossing and turning about the cushion. In response, the processor may activate an adjustment mechanism or cause the adjustment mechanism to adjust one or more characteristics of at least a portion of the cushion. Adjusting the one or more characteristics of at least a portion of the cushion may include causing air to flow through or across at least a portion of the sleeping surface 11. Additionally and/or alternatively, adjusting the one or more characteristics of at least a portion of the cushion 10 may include contouring at least a portion of the sleeping surface 11. Adjusting the one or more characteristics of at least a portion of the cushion may alleviate restless sleep and/or provide a neutral temperature for the user sleeping on the cushion.

Although this disclosure provides many specifics, these should not be construed as limiting the scope of any of the claims that follow, but merely as providing illustrations of some embodiments of elements and features of the disclosed subject matter. Other embodiments of the disclosed subject matter, and of their elements and features, may be devised which do not depart from the spirit or scope of any of the claims. Features from different embodiments may be employed in combination. Accordingly, the scope of each claim is limited only by its plain language and the legal equivalents thereto.

What is claimed:

1. A method of detecting movement during sleep, comprising:
monitoring a humidity at a sleeping surface over a duration of time; and
determining movement on the sleeping surface based on a change in humidity.

2. The method of claim 1, further comprising:
determining a position of a person resting on the sleeping surface based on a humidity at the sleeping surface.

3. The method of claim 1, wherein the at least one humidity sensor comprises a first humidity sensor located at a first location on the sleeping surface and a second humidity sensor located at a second location on the sleeping surface.

4. The method of claim 3, wherein the first location is a region where a leg and/or a foot of a user will rest on the sleeping surface.

5. The method of claim 4, wherein the second location is another region where a leg and/or a foot of the user will rest on the sleeping surface.

6. The method of claim 1, further comprising:
adjusting one or more characteristics of the sleeping surface based on the change in humidity.

7. The method of claim 6, wherein adjusting the one or more characteristics of the sleeping surface comprises adjusting a contour of the sleeping surface.

8. The method of claim 6, wherein adjusting one or more characteristics of the sleeping surface comprises adjusting a flow of air across and/or through at least a portion of the sleeping surface.

9. The method of claim 8, wherein adjusting the flow of air comprises adjusting the flow of air through the sleeping surface.

10. The method of claim 6, wherein adjusting one or more characteristics of the sleeping surface comprises adjusting a characteristic at a first location of the sleeping surface.

11. The method of claim 6, wherein adjusting one or more characteristics of the sleeping surface comprises adjusting a temperature of at least a portion of the sleeping surface.

12. The method of claim 6, wherein adjusting one or more characteristics of the sleeping surface comprises adjusting a firmness of at least a portion of the sleeping surface.

13. A method of adjusting a mattress during sleep, comprising:
capturing a plurality of humidity measurements at a sleeping surface over a period of time; and
adjusting one or more characteristics of at least a portion of the sleeping surface to reduce a type of movement by an individual indicated by the plurality of humidity measurements.

14. The method of claim 13, further comprising:
determining a spatial position of the individual resting on the sleeping surface based on the plurality of humidity measurements.

15. The method of claim 13, wherein adjusting the one or more characteristics of at least the portion of the sleeping surface comprises adjusting one or more of a contour, an air flow across and/or through, a temperature, and a firmness of the sleeping surface.

16. The method of claim 13, wherein adjusting the one or more characteristics of at least a portion of the sleeping surface comprises adjusting one or more characteristics at a first region of the sleeping surface and/or a second region of the sleeping surface, the second region being adjacent to the first region.

17. The method of claim 13, further comprising:
detecting a spike in humidity based on the plurality of humidity measurements over the period of time.

18. The method of claim 13, further comprising:
detecting a presence of the individual on the sleeping surface based on the plurality of humidity measurements.

19. The method of claim 18, wherein detecting the presence of the individual on the sleeping surface comprises identifying a spike in humidity over the period of time.

20. A smart sleeping system comprising:
a sleeping surface;
at least one humidity sensor associated with the sleeping surface; and
at least one processor in communication with the at least one humidity sensor to receive and process humidity measurements obtained with the at least one humidity sensor detect movement of an individual on the sleeping surface.

21. The smart sleeping system of claim 20, comprising a plurality of humidity sensors associated with the sleeping surface.

22. The smart sleeping system of claim 21, wherein the plurality of humidity sensors are associated with a leg region and/or a foot region of the sleeping surface.

23. The smart sleeping system of claim 20, further comprising:
an adjustment mechanism under control of the at least one processor based on the movement detected by the at least one processor.
